(19) United States
(12) Patent Application Publication
(10) Pub. No.: US 2013/0224262 A1
(43) Pub. Date: Aug. 29, 2013

(54) TRANSDERMAL PREPARATION

(71) Applicants: Mitsuji Akazawa, Kagawa (JP); Keiko Yamasaki, Kagawa (JP)

(72) Inventors: Mitsuji Akazawa, Kagawa (JP); Keiko Yamasaki, Kagawa (JP)

(73) Assignees: MEDRX CO., LTD., Higashikagawa-shi, Kagawa (JP); KM TRANSDERM, Osaka-shi, Osaka (JP)

(21) Appl. No.: 13/820,704

(22) PCT Filed: Sep. 16, 2010
(86) PCT No.: PCT/JP2010/005669

§ 371 Date: May 10, 2013

(30) Foreign Application Priority Data
Sep. 3, 2010 (JP) 2010-198373

(51) Int. Cl. A61K 31/167; A61K 9/00

(52) U.S. Cl. CPC A61K 31/167; A61K 9/0014
USPC 424/400; 514/626

(57) ABSTRACT

The invention provides a transdermal absorption preparation showing sufficient adhesiveness on adhesion to the skin, causing low skin irritation, and capable of well controlling releaseability of lidocaine. The transdermal absorption preparation contains a support and an adhesive layer formed on the support. The adhesive layer contains a drug, as well as a thermoplastic elastomer, more than 300 parts by weight of liquid paraffin per 100 parts by weight of the elastomer, not more than 10 wt % of a tackifier, lidocaine, and an organic acid.

### TECHNICAL FIELD

The present invention relates to a transdermal absorption preparation. More particularly, the present invention relates to a transdermal absorption preparation comprising lidocaine dispersed or dissolved together with an organic acid in liquid paraffin in a base, which causes low skin irritation and controlled releaseability.

### BACKGROUND ART

When a drug is to be transdermally absorbed, the drug is added to an adhesive base and the like and formed as an adhesive preparation. In recent years, tapes more superior in the adhesiveness are often used than poultices containing a large amount of water as a constituent component in an adhesive preparation. As an adhesive base for such tapes, a lipophilic adhesive base such as of rubber, acrylic or silicon type and the like is used. Of these, a rubber adhesive base is widely used since additives can be easily blended as compared to other adhesive bases. (patent documents 1-3)

On the other hand, as a transdermal absorption preparation containing lidocaine, poultices for herpetic neuralgia and postherpetic neuralgia (patent document 4), and tapes using rubber adhesive or acrylic adhesive for topical anesthesia (patent documents 5-7) are known.

However, problems have been pointed out for a transdermal absorption preparation using a rubber adhesive base such as high releaseability of lidocaine which makes the preparation inapplicable to herpetic neuralgia and postherpetic neuralgia, occurrence of skin irritation caused by a tackifier generally added to a transdermal absorption preparation and the like.

### DOCUMENT LIST

### Patent Documents

patent document 1: JP-A-2001-302502
patent document 2: JP-A-9-291028
patent document 3: JP-A-10-316559
patent document 4: JP-B-3115625
patent document 5: JP-B-2849950
patent document 6: JP-A-2000-319168
patent document 7: WO 2009/060629

### Non-Patent Document

non-patent document 1: “Nenchaku seihin no oyo gijutsu (Applied technology of adhesive product)” pp. 125-141, Keiji Fukuzawa et al., CMC Publishing Co., Ltd., 2000

### SUMMARY OF THE INVENTION

### Problems to be Solved by the Invention

An object of the present invention is to provide a transdermal absorption preparation, which has sufficient adhesiveness, causes low skin irritation, and shows controlled releaseability of lidocaine.

### Means of Solving the Problems

The present inventors have conducted intensive studies in an attempt to solve the aforementioned problems and found that a transdermal absorption preparation, which has sufficient adhesiveness and causes low skin irritation, can be obtained even without using a tackifier by using, as an adhesive base, a thermoplastic elastomer and a large amount of liquid paraffin relative to the elastomer, and that a transdermal absorption preparation of lidocaine, which shows controlled transdermal absorbability, can be obtained by adding an organic acid together with lidocaine, which resulted in the present invention. Particularly, it has been reported that a basic drug such as lidocaine forms an ion pair (organic acid salt) with an organic acid, which remarkably improves transdermal absorbability (WO 96/16642, WO 01/007018). However, it has been found that, when not less than an equimolar amount of an organic acid is contained, the transdermal absorbability of lidocaine in the present invention monotonically decreases conversely as the amount of the organic acid increases. Based on these results, the present inventors have conducted intensive studies and completed the present invention.

Accordingly, the gist of the present invention is as m follows.

(1) A transdermal absorption preparation comprising a support and an adhesive layer containing a drug and formed on the support, wherein the aforementioned adhesive layer comprises at least a thermoplastic elastomer, and more than 300 parts by weight of liquid paraffin per 100 parts by weight of the elastomer, and the adhesive layer comprises not more than 10 wt % of a tackifier, and lidocaine and an organic acid.
(2) The transdermal absorption adhesive preparation of the above-mentioned (1), wherein the content of the liquid paraffin in the adhesive layer is not less than 60 wt %.
(3) The transdermal absorption adhesive preparation of the above-mentioned (1) or (2), wherein the thermoplastic elastomer is a styrene-based block copolymer.
(4) The transdermal absorption adhesive preparation of the above-mentioned (3), wherein the styrene-based block copolymer is a styrene-isoprene-styrene copolymer.
(5) The transdermal absorption adhesive preparation of any of the above-mentioned (1)-(4), wherein the adhesive layer does not contain a tackifier.
(6) The transdermal absorption adhesive preparation of any of the above-mentioned (1)-(5), wherein the organic acid is at least one selected from aromatic sulfonic acid, aliphatic sulfonic acid, aromatic carboxylic acid and aliphatic carboxylic acid.
(7) The transdermal absorption adhesive preparation of any of the above-mentioned (1)-(6), wherein the organic acid has a content of 0.5- to 5-fold molar amount relative to lidocaine as 1.
(8) The transdermal absorption adhesive preparation of any of the above-mentioned (1)-(7), wherein the organic acid is at least one selected from lactic acid and isostearic acid.
(9) The transdermal absorption adhesive preparation of any of the above-mentioned (1)-(8), wherein the organic acid has a content of 0.8- to 3-fold molar amount relative to lidocaine as 1.
(10) The transdermal absorption adhesive preparation of any of the above-mentioned (1)-(9), wherein the transdermal absorption adhesive preparation is used for postherpetic neuralgia.
(11) The transdermal absorption adhesive preparation of the above-mentioned (10), wherein the organic acid has a content of 1.3- to 2-fold molar amount relative to lidocaine as 1.

### Effect of the Invention

The transdermal absorption preparation of the present invention has sufficient adhesiveness and causes low skin irritation when adhered to the skin, and can also well control releaseability of lidocaine.

### BRIEF DESCRIPTION OF THE DRAWINGS

FIG. 1 is a chart showing the comparison of the transdermal absorbability between the adhesive preparation of Example of the present invention, and that of Comparative Example.

### DESCRIPTION OF EMBODIMENTS

The transdermal absorption preparation of the present invention is characterized in that it has an adhesive layer containing a drug, which is formed on a support, wherein the aforementioned adhesive layer contains at least a thermoplastic elastomer, and more than 300 parts by weight of liquid paraffin per 100 parts by weight of the elastomer, and not more than 10 wt % of a tackifies in the adhesive layer, and also contains lidocaine and an organic acid.

The “thermoplastic elastomer” in the present invention is a thermoplastic elastomer having a hard segment and a soft segment, and examples thereof include various thermoplastic elastomers of urethane-based, acrylic, styrene-based or olefin-based type and the like. Of these, a styrene-based thermoplastic elastomer, particularly, a styrene-based block copolymer is preferably used for simultaneous achievement of sufficient adhesiveness and low skin irritation, which is the object of the present invention.

Specific examples include styrene-butadiene block copolymer, styrene-butadiene-styrene block copolymer, styrene-isoprene block copolymer, styrene-isoprene-styrene block copolymer, styrene-ethylene/butylene block copolymer, styrene-ethylene/butylene-styrene block copolymer, styrene-ethylene/propylene block copolymer, styrene-ethylene/propylene-styrene block copolymer, styrene-isobutylene block copolymer, styrene-isobutylene-styrene block copolymer and the like. Of these, only one kind of a styrene-based block copolymer may be used, or two or more kinds thereof may be used in combination.

Among these styrene-based block copolymers, a styrene-isoprene-styrene block copolymer, a styrene-isoprene block copolymer, and a mixture thereof are particularly preferably used to simultaneously achieve sufficient adhesiveness and low skin irritation, and in view of the availability and handling property for adhesion to the skin.

The “liquid paraffin” of the present invention is not particularly limited and a known commercially available product can be used.

As mentioned above, more than 300 parts by weight of liquid paraffin is added per 100 parts by weight of the thermoplastic elastomer in the present invention. As long as this ratio is satisfied, specific amounts of the thermoplastic elastomer and the liquid paraffin in the adhesive layer are not particularly limited. Generally, when the amount of the thermoplastic elastomer is too small, the form of an adhesive is difficult to maintain, and when it is too much, sufficient adhesiveness cannot be achieved. On the other hand, when the amount of the liquid paraffin is too small, sufficient adhesiveness is not obtained, and when it is too much, the form of an adhesive is difficult to maintain. Therefore, the upper limit of the content of liquid paraffin does not exceed 1500 parts by weight, since it influences adhesiveness and elasticity of the preparation. A preferable amount of liquid paraffin is 300-1000 parts by weight per 100 parts by weight of the elastomer.

The lower limit of the thermoplastic elastomer content is generally 5 wt %, preferably 8 wt %, more preferably 10 wt %. The upper limit is generally 25 wt %, preferably 20 wt %. The lower limit of the liquid paraffin content is generally 60 wt %, preferably 65 wt %, more preferably 70 wt %, particularly preferably 75 wt %. The upper limit is generally 95 wt %, preferably 90 wt %.

The transdermal absorption preparation of the present invention can exhibit good adhesiveness even when a tackifier is not added, by adopting the above-mentioned amounts of the thermoplastic elastomer and the liquid paraffin. The “tackifier” here means tackifier s generally used widely in the field of adhesive preparation. Examples thereof include rosin-based resin, polyterpene resin, cumarone-indene resin, petroleum-based resin, terpene-phenol resin, alicyclic saturated hydrocarbon resin and the like.

In the present invention, the content of the tackifier in the adhesive layer is not more than 10 wt % to reduce skin irritation and the like. It is preferably not more than 5 wt %, more preferably not more than 2 wt %, still more preferably not more than 1 wt %. Most preferably, no tackifier is contained. In relation to the adhesiveness of the preparation, moreover, the content of the tackifier is adjusted according to the amounts of the elastomer and liquid paraffin to be added and the ratios thereof. From such aspect, a preferable content of the tackifier is 0-5 wt %.

Lidocaine in the present invention refers to lidocaine or a salt thereof used for transdermal absorption. In view of availability, lidocaine or lidocaine hydrochloride is preferably used, and lidocaine is particularly preferably used in view of dispersibility in an adhesive and the like. While the content of lidocaine in the preparation is not particularly limited, it is preferably 1-10 wt %, particularly preferably 3-7 wt %, when application to herpetic neuralgia and postherpetic neuralgia is considered.

The “organic acid” of the present invention is not particularly limited and refers to aliphatic monocarboxylic acid, aliphatic dicarboxylic acid, aromatic carboxylic acid, organic sulfonic acid and the like. Examples of the aliphatic monocarboxylic acid include short-chain fatty acid having 2-7 carbon atoms such as acetic acid, butyric acid, hexanoic acid, cyclohexanecarboxylic acid and the like, medium-chain fatty acid having 8-11 carbon atoms such as octanoic acid, decanoic acid and the like, long-chain fatty acid having 12 or more carbon atoms such as myristic acid, stearic acid, isostearic acid, oleic acid and the like, short-chain fatty acid substituted by hydroxyl group, alkoxy group or acyl group, such as glycolic acid, lactic acid, methoxyacetic acid, mandelic acid, levulinic acid, 3-hydroxybutyric acid and the like, and the like. Examples of the aliphatic dicarboxylic acid include sebacic acid, adipic acid, malic acid, maleic acid, fumaric acid and the like.

Examples of the aromatic carboxylic acid include substituted or unsubstituted aromatic carboxylic acid such as benzoic acid, p-oxybenzoic acid, salicylic acid, acetylsalicylic acid, cinnamic acid and the like. Examples of the organic sulfonic acid include alkylsulfonic acid such as methanesulfonic acid, ethanesulfonic acid, menthylsulfonic acid and the like, aromatic sulfonic acid such as benzenesulfonic acid, toluenesulfonic acid, dodecylbenzenesulfonic acid and the like.

Preferable organic acid is, for example, lactic acid or isostearic acid, from the aspects of handling, availability, controllability of transdermal absorbability of lidocaine, adhesive property and the like.

While the amount of an organic acid to be added is not particularly limited, 0.5- to 5-fold molar amount of an organic acid is preferably added relative to lidocaine as 1. More preferably, 0.8- to 3-fold molar amount of an organic acid is added relative to lidocaine as 1. When the amount of an organic acid to be added is within the above-mentioned range, the skin permeability of lidocaine changes along with the amount of the organic acid to be added. Furthermore, when an organic acid is contained in not less than the equimolar amount, the transdermal absorbability can be controlled by adding 1- to 2-fold molar amount of the organic acid relative to lidocaine as 1, since the skin permeability of lidocaine monotonically decreases as the amount of the organic acid to be added increases. For example, when the preparation of the present invention (lidocaine 5%) is applied to herpetic neuralgia or postherpetic neuralgia, 1.3- to 2-fold molar amount of an organic acid is preferably added relative to lidocaine as 1 to ensure equivalence to commercially available lidocaine adhesive preparation A (lidocaine 5%). In addition, when the preparation of the present invention (lidocaine 5%) is used for topical anesthesia, 0.8- to 1.2-fold molar amount of an organic acid is preferably added relative to lidocaine as 1 to ensure equivalence to the effect of commercially available lidocaine adhesive preparation C (lidocaine 10%).

The transdermal absorption preparation of the present invention is constituted by extending an adhesive layer having the above-mentioned constitution on a support.

The “support” in the present invention is not particularly limited and those used widely can be employed. For example, stretchable or non-stretchable woven fabric or non-woven fabric of polyethylene, polypropylene and the like, films of polyethylene, polypropylene, ethylene vinyl acetate copolymer, vinyl chloride and the like, foamed supports of urethane, polyurethane and the like can be mentioned. These may be used alone, or plural kinds thereof may be laminated and uses. To prevent accumulation of static electricity on the support, as well as to achieve good anchor property to the adhesive layer, a non-woven fabric or woven fabric containing an antistatic agent can be used.

Moreover, the transdermal absorption preparation of the present invention may contain excipient, antioxidant, softener, flavor, colorant and the like as an optional component.

Examples of the excipient to be used in the present invention include silicon compounds such as silicic anhydride, light anhydrous silicic acid, hydrated silicate and the like, cellulose derivatives such as ethylcellulose, methylcellulose, hydroxypropyldellulose, hydroxypropylmethylcellulose and the like, water-soluble polymers such as polyvinyl alcohol and the like, aluminum compounds such as dried aluminum hydroxide gel, hydrated aluminum silicate and the like, kaolin, titanium oxide and the like.

Examples of the antioxidant to be used in the present invention include dibutylhydroxytoluene, ascorbic acid, tocopherol, tocopherol ester derivative, butylhydroxyanisole, 2-mercaptobenzimidazole and the like.

### EXAMPLES

The present invention is explained in more detail in the following by referring to Example and Comparative Examples, which are not to be construed as limitative.

### Example 1

Preparation of Transdermal Absorption Adhesive Preparation Containing Lidocaine and Organic Acid

Each agent is weighed to achieve the composition (w/w %) of the following Table 1, a styrene-isoprene-styrene copolymer is added to liquid paraffin, and the mixture is dissolved by heating to about 160° C. The solution is cooled to 100° C., a solution of lidocaine in organic acid is added and the mixture is mixed and stirred to give an adhesive base.

The adhesive base is applied to a silicon-treated polyester film, and adjusted to an amount of 1000 g/m². A polyester non-woven fabric is laminated on the surface of the adhesive base. This was cut in a desired size to give the object transdermal absorption adhesive preparation.

TABLE 1

| agent | No. 1 | No. 2 | No. 3 | No. 4 | No. 5 |
|---|---|---|---|---|---|
| elastomer: | 20 | 20 | 20 | 20 | 20 |
| styrene-isoprene- | | | | | |
| styrene | | | | | |
| copolymer | | | | | |
| liquid paraffin | 73 | 72 | 71.5 | 71 | 64 |
| organic acid: | | | | | |
| lactic acid | 2 | 3 | 3.5 | 4 | |
| isostearic acid | | | | | 11 |
| molar equivalent | 1.0 | 1.6 | 1.8 | 2.1 | 1.8 |
| of organic acid | | | | | |
| relative to | | | | | |
| lidocaine as 1 | | | | | |
| lidocaine | 5 | 5 | 5 | 5 | 5 |

The transdermal absorption preparation prepared in the above-mentioned Table 1 showed all good feeling and good adhesive property to the skin.

### Comparative Example 1

### Commercially Available Lidocaine Adhesive Preparation a (for Postherpetic Neuralgia)

An adhesive preparation containing 5.0 w/w % of lidocaine in an aqueous base composed of water, water-soluble polymer, polyhydric alcohol and the like was used. The base weight was 1000 g/m².

### Comparative Example 2

### Commercially Available Lidocaine Adhesive Preparation B (for Topical Anesthesia)

An adhesive preparation containing 60 wt % of lidocaine in an acrylic acid-octyl acrylate ester copolymer was used. The base weight was 19.6 g/m².

### Comparative Example 3

### Commercially Available Lidocaine Adhesive Preparation C (For Topical Anesthesia)

An adhesive preparation containing 10 wt % of lidocaine in a base composed of styrene-isoprene-styrene copolymer, alicyclic saturated hydrocarbon resin, liquid paraffin and the like was used. The base weight was 110 g/m².

### Experimental Example

### Transdermal Absorbability Evaluation Test of Adhesive Preparation

According to a known method (patent document 7 etc.), the abdominal skin of male Wister rat (5-week-old) was set on a vertical Franz diffusion cell, and test adhesive preparations of Example 1 and Comparative Examples 1-3 were each punched out in a circular shape (diameter 1.0 cm) and adhered to the rat skin of the diffusion cells (n=3). For the receptor, 10% ethanol saline was used, and the amount of drug that permeated the rat skin after a given time was quantified by HPLC.

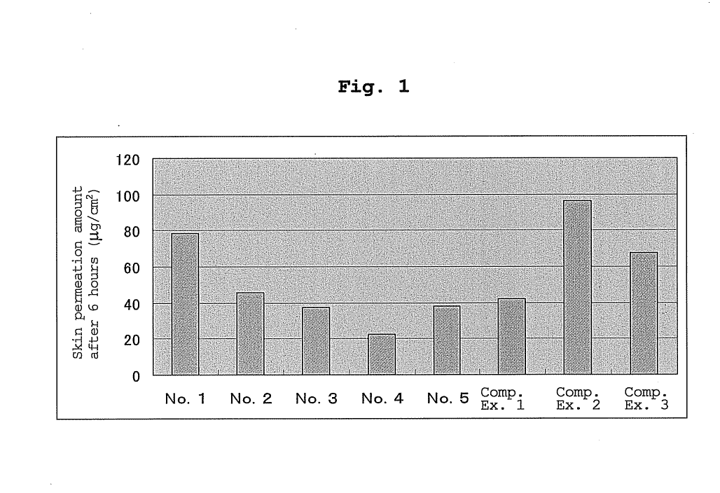

The results thereof (permeation amount of lidocaine at 6 hr after adhesion) are shown in FIG. 1. As is clear from FIG. 1, it was shown that the transdermal absorption preparation of the present invention can control transdermal absorbability of lidocaine by the amount of an organic acid to be added. As a result, the transdermal absorption preparation of the present invention obtained showed transdermal absorbability equivalent to not only that of a commercially available product for topical anesthesia but also of a commercially available product for postherpetic neuralgia (Comparative Example 1).

### INDUSTRIAL APPLICABILITY

The transdermal absorption preparation of the present invention is superior in the feeling on adhesion to the skin, can control transdermal absorbability of lidocaine, and may be utilizable for the improvement of the property of the existing transdermal absorption preparations and the development of a new transdermal absorption preparation.

1. A transdermal absorption preparation comprising a support and an adhesive layer containing a drug and formed on the support, wherein the adhesive layer comprises a thermoplastic elastomer, more than 300 parts by weight of liquid paraffin per 100 parts by weight of the elastomer, lidocaine, and an organic acid, and optionally comprises not more than 10 wt % of a tackifier.

2. The transdermal absorption adhesive preparation according to claim 1, wherein the content of the liquid paraffin in the adhesive layer is not less than 60 wt %.

3. The transdermal absorption adhesive preparation according to claim 1, wherein the thermoplastic elastomer is a styrene-based block copolymer.

4. The transdermal absorption adhesive preparation according to claim 3, wherein the styrene-based block copolymer is a styrene-isoprene-styrene copolymer.

5. The transdermal absorption adhesive preparation according to claim 1, wherein the adhesive layer does not contain a tackifier.

6. The transdermal absorption adhesive preparation according to claim 1, wherein the organic acid is at least one selected from aromatic sulfonic acid, aliphatic sulfonic acid, aromatic carboxylic acid and aliphatic carboxylic acid.

7. The transdermal absorption adhesive preparation according to claim 1, wherein the organic acid has a content of 0.5- to 5-fold molar amount relative to lidocaine as 1.

8. The transdermal absorption adhesive preparation according to claim 1, wherein the organic acid is at least one selected from lactic acid and isostearic acid.

9. The transdermal absorption adhesive preparation according to claim 1, wherein the organic acid has a content of 0.8- to 3-fold molar amount relative to lidocaine as 1.

10. The transdermal absorption adhesive preparation according to claim 1, wherein the transdermal absorption adhesive preparation is used for postherpetic neuralgia.

11. The transdermal absorption adhesive preparation according to claim 10, wherein the organic acid has a content of 1.3- to 2-fold molar amount relative to lidocaine as 1.

12. The transdermal absorption adhesive preparation according to claim 2, wherein the thermoplastic elastomer is a styrene-based block copolymer.

13. The transdermal absorption adhesive preparation according to claim 12, wherein the styrene-based block copolymer is a styrene-isoprene-styrene copolymer.

14. The transdermal absorption adhesive preparation according to claim 13, wherein the adhesive layer does not contain a tackifier.

15. The transdermal absorption adhesive preparation according to claim 14, wherein the organic acid is at least one selected from aromatic sulfonic acid, aliphatic sulfonic acid, aromatic carboxylic acid and aliphatic carboxylic acid.

16. The transdermal absorption adhesive preparation according to claim 15, wherein the organic acid has a content of 0.5- to 5-fold molar amount relative to lidocaine as 1.

17. The transdermal absorption adhesive preparation according to claim 16, wherein the organic acid is at least one selected from lactic acid and isostearic acid.

18. The transdermal absorption adhesive preparation according to claim 17, wherein the organic acid has a content of 0.8- to 3-fold molar amount relative to lidocaine as 1.

19. The transdermal absorption adhesive preparation according to claim 18, wherein the transdermal absorption adhesive preparation is used for postherpetic neuralgia.

20. The transdermal absorption adhesive preparation according to claim 19, wherein the organic acid has a content of 1.3- to 2-fold molar amount relative to lidocaine as 1.
